(19) United States
(12) Patent Application Publication
(10) Pub. No.: US 2020/0114015 A1
(43) Pub. Date: Apr. 16, 2020

(54) Novel Alpha-Hydroxy Carboxylic Acid And Derivatives And Other GRAS- Based Prodrugs Of Gamma-Hydroxybutyrate (GHB) And Uses Thereof

(71) Applicant: John K. Thottathil, Mundelein, IL (US)

(72) Inventor: John K. Thottathil, Mundelein, IL (US)

(21) Appl. No.: 16/603,880

(22) PCT Filed: Apr. 10, 2018
(86) PCT No.: PCT/US18/26821

§ 371 Date: Oct. 9, 2019

Related U.S. Application Data
(60) Provisional application No. 62/484,024, filed on Apr. 11, 2017.

(51) Int. Cl. A61K 47/54; A61K 31/19

(52) U.S. Cl. CPC A61K 47/542; A61K 31/19

(57) ABSTRACT

The invention describes pharmaceutical compounds and compositions comprised of prodrug ligands attached to GHB (CNS drugs) in a manner that substantially decreases or deters the potential for GHB abuse, illicit and illegal use, and overdose. These compounds and compositions may provide substantially higher bioavailability, substantially higher half-life, substantially higher chemical and biological stability, and easier shipping and distribution requirements. These GHB prodrug compounds may alter both the physical and chemical properties and thus may not be suitable for illicit use as a date-rape drug (i.e., they may not dissolve instantly in water based drinks, may have different color once in solution, and/or may have a taste and odor once in solution etc.). When delivered at the proper dosage, the pharmaceutical composition provides therapeutic activity similar to that of the parent active agent GHB.

### CROSS REFERENCE TO RELATED APPLICATIONS

This application claims the benefit of U.S. Provisional Application No. 62/484,024, filed Apr. 11, 2017.

### FIELD OF INVENTION

The present invention relates to pharmaceutical compounds, compositions, and methods of using chemical moieties that are generally recognized as safe (GRAS), which are attached to gamma-hydroxybutyrate (GHB), also known as 4-hydroxybutyrate. These chemical moieties include monomers, as well as homo- and hetero-oligomers, of alpha-hydroxy carboxylic acids and their chemical derivatives. The invention relates to GHB covalently bound to, or conjugated to, the chemical moieties. The present invention relates generally to pharmaceutical compounds to be used in treatments, such as, sleeping disorders, e.g., narcolepsy (particularly cataplexy), drug abuse, alcohol and opiate withdrawal, a reduced level of growth hormone, anxiety, analgesia, effects in certain neurological disorders such as Parkinson's Disease, depression, certain endocrine disturbances and tissue protection following hypoxia/anoxia such as in stroke or myocardial infarction, or for an increased level of intracranial pressure or the like.

The present invention provides a variety of beneficial effects; convenience of dosing, lower quantities of dosing, increased unit dose bioavailability, a substantial decrease in the potential of GHB to cause overdose or to be abused, especially as a causative factor in date-rape. Some embodiments of the invention provide therapeutic activity similar to that of unmodified GHB at typical dosage ranges, but when delivered at higher doses, the potential for overdose is reduced due to decreased bioavailability of GHB, especially when taken by non-approved routes, as compared to GHB that is administered by the approved oral route and delivered in a non-conjugated form. Additionally, these prodrugs may be designed to provide fast or slow release of GHB depending on its standard use for various CNS ailments.

### BACKGROUND OF THE INVENTION

GHB is an endogenous compound with hypnotic properties that is found in many human body tissues. GHB is present, for example, in the mammalian brain and other tissues. In brain the highest GHB concentration is found in the hypothalamus and basal ganglia and GHB is postulated to function as a neurotransmitter (Snead and Morley, 1981). The neuropharmacologic effects of GHB include increases in brain acetylcholine, increases in brain dopamine, inhibition of GABA-ketoglutarate transaminase and depression of glucose utilization but not oxygen consumption in the brain. GHB is converted to succinate and then metabolized via the Krebs cycle. Clinical trials have shown that GHB increases delta sleep and improves the continuity of sleep (Ladinsky et al., 1983; Anden and Stock, 1973; Stock et al., 1973; Laborit, 1973; Lapierre et al., 1988; Lapierre et al., 1990; Yamda et al., 1967; Grove-White and Kelman, 1971; Scharf, 1985).

GHB has typically been administered in clinical trials as an oral solution (Lee, 1977; Mamelak, 1977; Hoes, 1980; Scharf, 1985; Scrima, 1990; Gallimberti, 1992; Series, 1992; Lammers, 1993). GHB treatment substantially reduces the signs and symptoms of narcolepsy, i.e. daytime sleepiness, cataplexy, sleep paralysis and hypnagogic hallucinations. In addition, GHB increases total sleep time and REM sleep (Rapid Eye Movement sleep), and it decreases REM latency (Mamelak et al, 1973; Yamada et al., 1967; Bedard et al., 1989), reduces sleep apnea (Series el al, 1992; Scrima et al., 1987), and improves general anesthesia (Hasenbos and Gielen, 1985).

GHB has several clinical applications other than narcolepsy and sleep disorders. GHB has been reported to reduce alcohol craving, the number of daily drinks consumed, and the symptoms of alcohol withdrawal in patients (Gallimberti et a., 1989; Gallimberti et al., 1992; Gessa et al., 1992). GHB has been used to decrease the symptoms of opiate withdrawal, including both heroin and methadone withdrawal (Gallimberti et al, 1994; Gallimberti et al., 1993). It has analgesic effects that make it suitable as a pain reliever (U.S. Pat. No. 4,393,236). Intravenous administration of GHB has been reported to reduce intracranial pressure in patients (Strong, A. 1984). Also, administration of GHB was reported to increase growth hormone levels in patients (Gerra et al, 1994; Oyama et al., 1970).

A good safety profile for GHB consumption, when used long term for treatment of narcolepsy, has been reported. Patients have been safely treated for many years with GHB without development of tolerance (Scharf, 1985). Clinical laboratory tests carried out periodically on many patients have not indicated organ or other toxicities (Lammers, 1993; Scrima, 1990; Scharf, 1985; Mamelack, 1977; Mamelak, 1979; Gallimberti, 1989; Gallimberti, 1992; Gessa, 1992). The side effects of GHB treatment have been minimal in incidence and degree of severity, though they include sleepwalking, enuresis, headache, nausea and dizziness (Broughton and Mamelak, 1979; Mamelak et al., 1981; Mamelak et al., 1977; Scrima et al., 1989; Scrima et al., 1990; Scharf et al., 1985).

The pharmacokinetics of GHB has been investigated in alcohol dependent patients (Ferrara et al., 1992) and in normal healthy males (Palatini et al., 1993) after oral administration. GHB possesses a rapid onset and short pharmacological effect (Ferrara et al., 1992; Palatine et al., 1993; Lee, C., 1977; van der Bogert; Gallimberti, 1989; Gallimberti, 1992; Lettieri and Fung, 1978; Arena and Fung, 1980; Roth and Giarman, 1966; Vickers, 1969; Lee, 1977). In alcohol dependent patients, GHB absorption into and elimination from the systemic circulation were fast processes. Virtually no unchanged drug could be recovered in the urine. There were preliminary indications that the pharmacokinetics of GHB might be non-linear or dose-dependent (Ferrara et al., 1992). In the healthy volunteers study, the pharmacokinetics of three rising GHB doses (12.5, 25, and 50 mg/kg) were investigated. These findings indicate that both the oral absorption and elimination processes of GHB were capacity-limited though the degree of dose dependency was moderate (Palatini et al., 1993).

Organic salts and amides of GHB have been produced to reduce the physiological side effects of GHB (U.S. Pat. No. 5,380,937). Magnesium and calcium salts have been produced to reduce the hygroscopic nature of GHB (U.S. Pat. No. 4,393,236; British Patent No. 922,029). However, problems with the storage of GHB solutions still exist. GHB degrades into gamma-butyrolactone (GBL) and possibly other degradants in solution depending upon the pH and other factors. Also, the contamination by microorganisms in GHB solutions rapidly surpasses acceptable limits, and preservatives can adversely affect the pH and thus, GHB's stability. As a chronically used product which requires high levels of drug, the volume of a non-concentrated product creates cost and handling issues.

GHB marketed as Xyrem® has low bioavailability (˜25%), short half life (30-60 minutes), has the infamous reputation as the date-rape drug and its physical properties are conducive to illicit use, as it dissolves very well, and very fast, in alcohols and common drinks and beverages with no color, or taste or odor. Xyrem® is the only drug to be on two DEA drug schedules at once. Under U.S. law, GHB is on schedule II (considered dangerous and addictive drug) but GHB marketed as Xyrem® and prescribed for specific conditions is on schedule III requiring tight controls for shipping, storage and distribution. Because of the short half life of the medication, patients who are taking Xyrem® as a sleep aid are required to wake-up half way through their sleep at night to take the second dose of the medicine to be effective for the treatment. Further, because of the high dose (4.5-9.0 gms) requirements due to low bioavailability, the medication put limits on how much quantity a patient can have in a given day.

Accidental and intentional overdose with prescription and/or over-the-counter (OTC) drugs is a serious health problem that is associated with thousands of fatalities every year. Accidental overdose can also commonly occur when unusually potent batches of illicit GHB are ingested by drug addicts or other abusers.

Thus, there is an immediate need for effective solutions to the issues of GHB compounds. To reduce the potential for overdose and to reduce or deter GHB substance abuse and to provide sustained release and sustained therapeutic effect and that are stable to biological or chemical degradation.

Prodrug chemistry has been tried and met with moderate success in the pharmaceutical industry especially in the case of such drugs as amphetamine (Vyvanse) and opioid pain medications.

Ideally, a prodrug moiety and its linkage to GHB would be cleaved at an appropriate rate and site, which would then release the active drug GHB into the blood and provide the intended therapeutic benefit while sharply reducing their principal limitations, including variable bioavailability after oral dosing, overdose, and misuse, illegal/illicit use and product tampering.

### SUMMARY

Provided are pharmaceutical compounds, compositions, and methods of using such compounds and compositions. Also provided are methods of using chemical moieties (prodrug moieties) that are generally recognized as safe (GRAS), which are attached to the GHB molecule to produce a conjugated GHB prodrug compound. These chemical moieties include monomers, homo- and hetero-oligomers of alpha-hydroxy carboxylic acids, and their chemical derivatives. The prodrug GHB compounds may provide a substantial decrease in the potential of GHB to cause overdose or to be abused. In some embodiments these prodrug conjugates provide therapeutic activity which is similar to that of unmodified parent drug GHB when delivered at typical dosage ranges. However, when delivered at higher doses the potential for overdose is reduced as compared to conventional non-conjugated GHB due to decreased bioavailability of the GHB, especially when taken by non-approved oral routes. The prodrugs may provide fast or slow release of GHB depending on its standard use for chronic or acute causes. Additionally, the prodrugs may be designed to provide substantially higher bioavailability, substantially higher half-life, substantially higher chemical and biological stability, and easier shipping and distribution requirements. These prodrugs may alter both the physical and chemical properties and thus may not be suitable for illicit use as a date rape drug (e.g., they may not dissolve instantly in water based drinks, may have different color once in solution, and/or may have a taste and odor once in solution etc.). These prodrugs may provide a smooth, sustained and controlled release of the active GHB in the plasma and thus have the potential for single dosing in the night.

The present invention provides methods to treat a number of conditions treatable by GHB, referred to herein as “therapeutic categories.” Therapeutic categories for the present invention include, but are not limited to, sleeping disorders, drug abuse, alcohol and opiate withdrawal, a reduced level of growth hormone, anxiety, analgesia, effects in certain neurological disorders, such as Parkinson's Disease, depression, certain endocrine disturbances and tissue protection following hypoxia/anoxia such as in stroke or myocardial infarction, or an increased level of intracranial pressure or other conditions treatable with GHB.

### DETAILED DESCRIPTION OF THE INVENTION

A first aspect of the invention relates to changing the pharmacokinetic, pharmacological, and physico-chemical properties of CNS drug GHB through covalent modification using alpha-hydroxy carboxylic acid and derivatives and other generally recognized as safe (GRAS)-based moieties to produce prodrugs of GHB. Covalent attachment of a chemical moiety—specifically, a moiety derived from alpha-hydroxy carboxylic acid and derivatives, and other GRAS-based reagents as monomers and oligomers (homo and hetero oligomers)—to GHB may change one or more of the following properties of GHB: the rate of absorption; extent of absorption and distribution within the body; metabolism and drug elimination (i.e., ADME pharmacokinetic properties) and its physico-chemical properties. As such, the alteration of one or more of these characteristics may be designed to provide fast or slow release of the parent drug, depending on need for relief of chronic versus acute CNS diseases. Additionally, alteration of one or more of these characteristics may reduce the previously noted side-effects associated with GHB. These prodrugs may alter both the physical and chemical properties of GHB, and thus may not be suitable for illicit users as a date-rape drug (e.g., it may not dissolve instantly in water-based drinks, may have different color once in solution, and/or may have a taste and odor once in solution etc.). In turn, these alterations in drug properties may diminish or deter abuse potential. The oligomers formed from alpha-hydroxy carboxylic acid and derivatives can be homo- or hetero-‘mers’, and can be either linear or branched ‘mers’. The hetero ‘mers’ can be cross linked with other GRAS reagents, such as other alpha-hydroxy carboxylic acid, amino acid and dicarboxylic acids including, but not limited to, fumaric acid, maleic acid and succinic acid.

The GHB prodrugs may also prevent abuse by exhibiting stability under conditions that are likely to be employed by chemists who may illicitly attempt to release the GHB compound from its attached prodrug group. The GHB prodrugs may further prevent abuse by exhibiting reduced bioavailability when administered via parenteral routes, particularly by intravenous, intranasal, or inhalation (“smoking”) routes that are often employed in illicit use. Thus, the GHB prodrugs may reduce the desired euphoric effect associated with GHB abuse. Thus, the GHB prodrug may prevent, deter, or reduce abuse potential and overdose when the GHB prodrug is used in an unapproved manner (e.g., ingestion at a higher dose or non-oral administration). The GHB prodrugs may have higher intrinsic bioavailability and thus require lower dosage quantities compared to the current levels (4.5-9.0 gms/day).

The GHB prodrugs may have longer half-life and longer periods of sustained blood concentration of the drug, and thus avoid the need to take a second dose of the medication half-way through the sleep of the patient in the night.

GHB prodrugs of the present invention may be depicted as Formula A, Formula B, or Formula C where “X” and “Y” represent the prodrug components that are chemically/covalently attached to the GHB molecule. The prodrug components “X” and “Y” can be any moiety that alters the pharmacokinetic, pharmacological, and physico-chemical properties of the GHB while bound to the parent molecule, as compared to unbound (free) GHB.

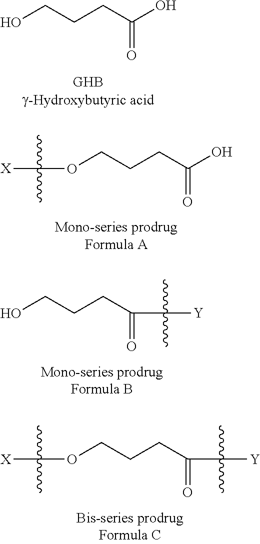

GHB prodrugs represented by formula A and Formula B represent the mono-series of prodrugs (i.e., GHB with a single prodrug moiety modification) while Formula C represents the bis-series of GHB prodrug (i.e., GHB with two prodrug moiety modifications). In the case of Formula A, the prodrug moiety X is chemically/covalently attached to the hydroxyl (—OH) group of GHB and it is an ester bond. In the case of Formula B, the prodrug moiety Y is chemically/covalently attached to the carboxylic acid group of GHB and it is either an ester or an amide bond depending on the functionality of the group that is attached to the carboxylic acid group of GHB. If the hydroxyl group from a prodrug moiety Y is attached to the carboxylic acid group of GHB, then it will be an ester bond, and if the amino group from a prodrug moiety Y is attached to the carboxylic acid group of GHB, then it will be an amide bond. Formula C is the bis-series of GHB prodrug wherein the prodrug moiety X is chemically/covalently attached to the hydroxyl (—OH) group of GHB via an ester bond and the prodrug moiety Y is chemically/covalently attached to the carboxylic acid group of GHB via an ester or an amide bond depending on the functionality of the group that is attached to the carboxylic acid group of GHB. Ligands X and Y may be the same or different.

Examples of the alpha-hydroxy carboxylic acids and other GRAS-based monomers used to make the monomer-based and oligomer-based GHB prodrugs, Formula A, Formula B, and Formula C are depicted below.

It should be emphasized that the following chemical moieties represent non-limiting examples of alpha-hydroxy carboxylic acids and other GRAS-based monomers used to make the monomer-based and oligomer-based GHB prodrugs of the present invention:

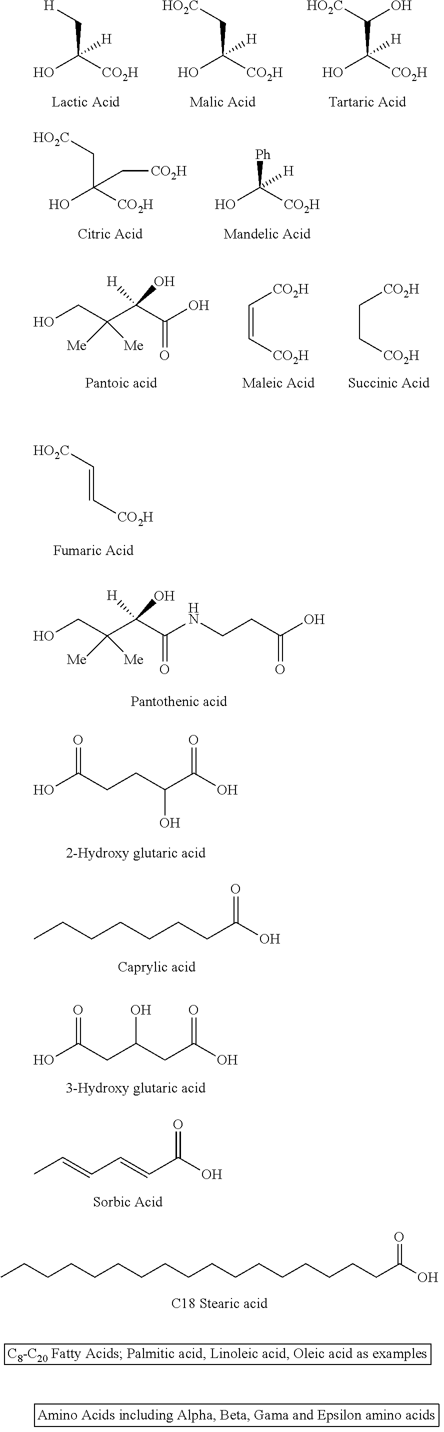

The alpha-hydroxy carboxylic acids represented here for use in the invention include the naturally occurring (L)-isomers, the non-natural (D)-isomers, mixtures of (L) and (D) isomers, racemates and mixtures of diastereomers, and meso-isomers. The term “alpha-hydroxy carboxylic acid” as used herein is intended to encompass any or all of the foregoing.

The amino acids represented here for use in the invention include both natural and non-natural amino acids, the naturally occurring (L)-isomers, the non-natural (D)-isomers, mixtures of (L) and (D) isomers, racemates and mixtures of diastereomers. The term “amino acid” as used herein is intended to encompass any or all of the foregoing. When reference is made to a “side chain” of an amino acid, it is intended that the side chain may be the side chain of any of the foregoing types of amino acid.

The amino acids represented here for use in the invention also include alpha amino acids, beta amino acids, gamma amino acids, and epsilon amino acids (i.e., amino group remote relative to the carboxyl group), and di-carboxylic acid amino acids such as aspartic acid and glutamic acid. The term “amino acid” as used herein is also intended to encompass any or all of the foregoing. When reference is made to a “side chain” of an amino acid, it is intended that the side chain may be the side chain of any of the foregoing types of amino acid.

The fatty acids represented here for use in the invention include long-chain carboxylic acids, ranging in carbon lengths from eight carbons (C8) to twenty carbons (C20). These fatty acids could be either linear or branched, and either saturated or non-saturated. In the case of unsaturated fatty acids, they could be either cis- or trans-isomers (Z and E isomers). The term “fatty acid” as used herein is intended to encompass any or all of the foregoing.

The alpha-hydroxy carboxylic acids and other GRAS-based monomers represented here are used to make the monomer-based and oligomer-based GHB prodrugs of Formula A, Formula B and Formula C.

In one embodiment of the present invention, the prodrug component X may be represented as,

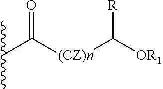

wherein,

### CZ═CH2, or CHOR1,

R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R=Methyl (Me), Phenyl (Ph), CH2COR2, CHOR1COR2, or COR2 (when n is not zero),
where R2=OH, an ester formed by the hydroxyl group of another alpha-hydroxy acid or an amide formed by the amine group of an amino acid, and,
n is an integer selected from 0 to 2.

In another embodiment of the present invention, the prodrug component X may be represented as,

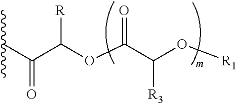

wherein,
R1=H, an acyl linkage of a fatty acid, an acyl linkage of an alpha-hydroxy acid, an acyl linkage of an amino acid or an acyl linkage of a dicarboxylic acid including, but not limited to, fumaric acid, maleic acid and succinic acid, and,
R and R3 can be same or different, and,
R and R3 are each independently Me, Ph, CH2COR2, or CHOR1COR2, where R2=OH or is an ester formed by the hydroxyl group of another alpha-hydroxy acid or is an amide formed by the amine group of an amino acid, and,
m is an integer selected from 0 to 4.

In another embodiment of the present invention, the prodrug component X may be represented as,

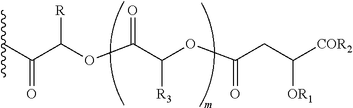

wherein,
R1=H, an acyl linkage of a fatty acid, an acyl linkage of an alpha-hydroxy acid, an acyl linkage of an amino acid or an acyl linkage of a dicarboxylic acid including, but not limited to, fumaric acid, maleic acid and succinic acid, and,
R and R3 can be same or different, and,
R and R3 are each independently Me, Ph, CH2COR2, or CHOR1COR2, where R2=OH or is an ester formed by the hydroxyl group of another alpha-hydroxy acid or is an amide formed by the amine group of an amino acid, and,
m is an integer selected from 0 to 4.

In another embodiment of the present invention, the prodrug component X may be represented as,

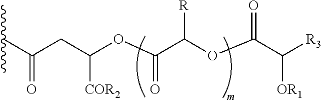

wherein,
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R and R3 can be same or different, and,
R and R3 are each independently Me, Ph, CH2COR2, or CHOR1COR2, where R2=OH or is an ester formed by the hydroxyl group of another alpha-hydroxy acid or is an amide formed by the amine group of an amino acid, and,
m is an integer selected from 0 to 4.

In another embodiment of the present invention, the prodrug component X may be represented as,

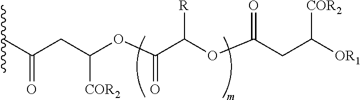

wherein,
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R=Me, Ph, CH2COR2, or CHOR1COR2, where R2=OH or is an ester formed by the hydroxyl group of another alpha-hydroxy acid or is an amide formed by the amine group of an amino acid, and,
m is an integer selected from 0 to 4.

In another embodiment of the present invention, the prodrug component X may be represented as,

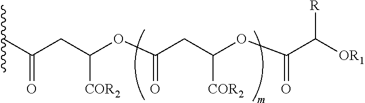

wherein,
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R=Me, Ph, CH2COR2, or CHOR1COR2, where R2=OH or is an ester formed by the hydroxyl group of another alpha-hydroxy acid or is an amide formed by the amine group of an amino acid, and,
m is an integer selected from 0 to 4.

In another embodiment of the present invention, the prodrug component X may be represented as,

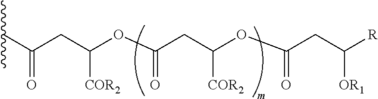

wherein,
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R=Me, Ph, CH2COR2, or CHOR1COR2, where R2=OH or is an ester formed by the hydroxyl group of another alpha-hydroxy acid or is an amide formed by the amine group of an amino acid, and,
m is an integer selected from 0 to 4.

In another embodiment of the present invention, the prodrug component X may be represented as,

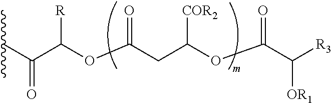

wherein,
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid and,
R and R3 can be same or different, and,
R and R3 are each independently Me, Ph, CH2COR2, or CHOR1COR2, where R2=OH or is an ester formed by the hydroxyl group of another alpha-hydroxy acid or is an amide formed by the amine group of an amino acid, and,
m is an integer selected from 0 to 4.

In another embodiment of the present invention, the prodrug component X may be represented as,

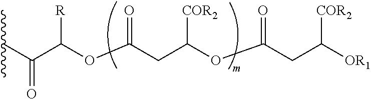

wherein,
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R=Me, Ph, CH2COR2, or CHOR1COR2, where R2=OH or is an ester formed by the hydroxyl group of another alpha-hydroxy acid or is an amide formed by the amine group of an amino acid, and,
m is an integer selected from 0 to 4

In another embodiment of the present invention, the prodrug component X may be represented as,

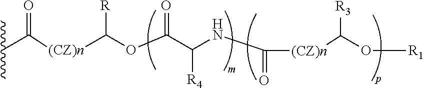

wherein,

### CZ═CH2, or CHOR1;

R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R and R3 can be same or different, and,
R and R3 are each independently Me, Ph, CH2COR2, or CHOR1COR2, where R2=OH or is an ester formed by the hydroxyl group of another alpha-hydroxy acid or is an amide formed by the amine group of an amino acid, and,
R4 is a side chain of an amino acid, and,
m is an integer selected from 0 to 4, and,
n is an integer selected from 0 to 2, and,
p is an integer selected from 0 to 1.

In another embodiment of the present invention, the prodrug component X may be represented as,

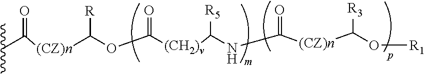

wherein,

### CZ═CH2, or CHOR1;

R1=H, an acyl linkage of a fatty acid, an acyl linkage of an alpha-hydroxy acid, an acyl linkage of an amino acid, and an acyl linkage of a dicarboxylic acid including, but not limited to, fumaric acid, maleic acid and succinic acid, and, R and R3 can be same or different, and,
R and R3 are each independently Me, Ph, CH2COR2, or CHOR1COR2, where R2=OH or is an ester formed by the hydroxyl group of another alpha-hydroxy acid or is an amide formed by the amine group of an amino acid, and,
R5=H, or COR2, where R2=OH or is an ester formed by the hydroxyl group of another alpha-hydroxy acid or is an amide formed by the amine group of an amino acid, or is an alkyl ester (O-alkyl, alkyl group is 1-4 carbon linear or branched, saturated or non-saturated alkyl groups), and,
m is an integer selected from 0 to 4, and,
n is an integer selected from 0 to 2, and,
p is an integer selected from 0 to 1, and,
v is an integer selected from 0 to 5.

In another embodiment of the present invention, the prodrug component X may be represented as,

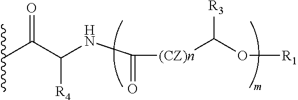

wherein,

### CZ═CH2, or CHOR1;

R1=H, an acyl linkage of a fatty acid, an acyl linkage of an alpha-hydroxy acid, an acyl linkage of a amino acids or an acyl linkage of a dicarboxylic acid including, but not limited to, fumaric acid, maleic acid and succinic acid, and,
R3=Me, Ph, CH2COR2, or CHOR1COR2, where R2=OH or is an ester formed by the hydroxyl group of another alpha-hydroxy acid or is an amide formed by the amine group of an amino acid, and,
R4 is the side chain of an amino acid, and,
m is an integer selected from 0 to 4, and,
n is an integer selected from 0 to 2,

In another embodiment of the present invention, the prodrug component X may be represented as,

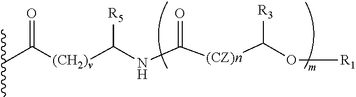

wherein,

### CZ═CH2, or CHOR1;

R1=H, an acyl linkage of a fatty acid, an acyl linkage of an alpha-hydroxy acids, an acyl linkage of an amino acid, or an acyl linkage of a dicarboxylic acid including, but not limited to, fumaric acid, maleic acid and succinic acid, and,
R3=Me, Ph, CH2COR2, or CHOR1COR2, where R2=OH or is an ester formed by the hydroxyl group of another alpha-hydroxy acid or is an amide formed by the amine group of an amino acid, and,
R5=H, or COR2, where R2=OH or is an ester formed by the hydroxyl group of another alpha-hydroxy acid or is an amide formed by the amine group of an amino acid, or is an alkyl ester (O-alkyl, alkyl group is 1-4 carbon linear and branched, saturated and non-saturated alkyl groups), and,
m is an integer selected from 0 to 4, and,
n is an integer selected from 0 to 2, and,
v is an integer selected from 0 to 5.

In another embodiment of the present invention, the prodrug component X may be represented as,

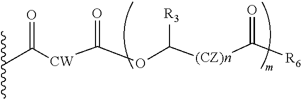

wherein,
CW═(CH2)q, or CH═CH (both E and Z isomers),

### CZ═CH2, or CHOR1;

R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R3=Me, Ph, CH2COR2, or CHOR1COR2, where R2=OH or is an ester formed by the hydroxyl group of another alpha-hydroxy acid or is an amide formed by the amine group of an amino acid, and,
R6=OH or is an ester formed by the hydroxyl group of another alpha-hydroxy acid or is an amide formed by the amine group of an amino acid, or is an alkyl ester (O-alkyl, alkyl group is 1-4 carbon linear or branched, saturated or non-saturated alkyl groups), and, m is an integer selected from 0 to 4, and,
n is an integer selected from 0 to 2, and,
q is an integer selected from 2 to 6.

In another embodiment of the present invention, the prodrug component X may be represented as,

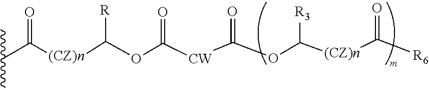

wherein,
CW═(CH2)q, or CH═CH (both E and Z isomers),

### CZ═CH2, or CHOR1;

R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R and R3 can be the same or different, and,
R and R3 are each independently Me, Ph, CH2COR2, or CHOR1COR2, where R2=OH or is an ester formed by the hydroxyl group of another alpha-hydroxy acid or is an amide formed by the amine group of an amino acid, and,
R6=OH or is an ester formed by the hydroxyl group of another alpha-hydroxy acid or is an amide formed by the amine group of an amino acid, or is an alkyl ester (O-alkyl, alkyl group is 1-4 carbon linear or branched, saturated or non-non-saturated alkyl groups), and
m is an integer selected from 0 to 4, and,
n is an integer selected from 0 to 2, and,
q is an integer selected from 2 to 6.

In another embodiment of the present invention, the prodrug components X may be represented as,

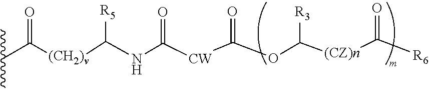

wherein,
CW═(CH2)q, or CH═CH, (both E and Z isomers), and,
R3=Me, Ph, CH2COR2, or CHOR1COR2, where R2=OH or is an ester formed by the hydroxyl group of another alpha-hydroxy acid or is an amide formed by the amine group of an amino acid,
R5=H, or COR2, where R2=OH or is an ester formed by the hydroxyl group of another alpha-hydroxy acid or is an amide formed by the amine group of an amino acid, or is an alkyl ester (O-alkyl, alkyl group is 1-4 carbon linear or branched, saturated or non-saturated alkyl groups), and,
R6=OH or is an ester formed by the alcohol (OH) of another alpha-hydroxy acid or is an amide formed by the amine group of an amino acid, or is an alkyl ester (O-alkyl, alkyl group is 1-4 carbon linear or branched, saturated or non-saturated alkyl groups), and,
m is an integer selected from 0 to 4, and,
n is an integer selected from 0 to 2, and,
q is an integer selected from 2 to 6, and
v is an integer selected from 0 to 6.

In another embodiment of the present invention, the prodrug component X may be represented as,

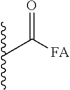

wherein,
FA is C8 to C20 saturated or unsaturated fatty acid including sorbic acid, stearic acid, oleic acid, palmitic acid, linoleic acid. These fatty acids may be linear or branched chain acids, or a combination thereof; and in the case of unsaturated fatty acids, they may be cis- or trans-isomers (Z and E isomers).

In another embodiment, the prodrug components Y may be represented any of Ligands A-N

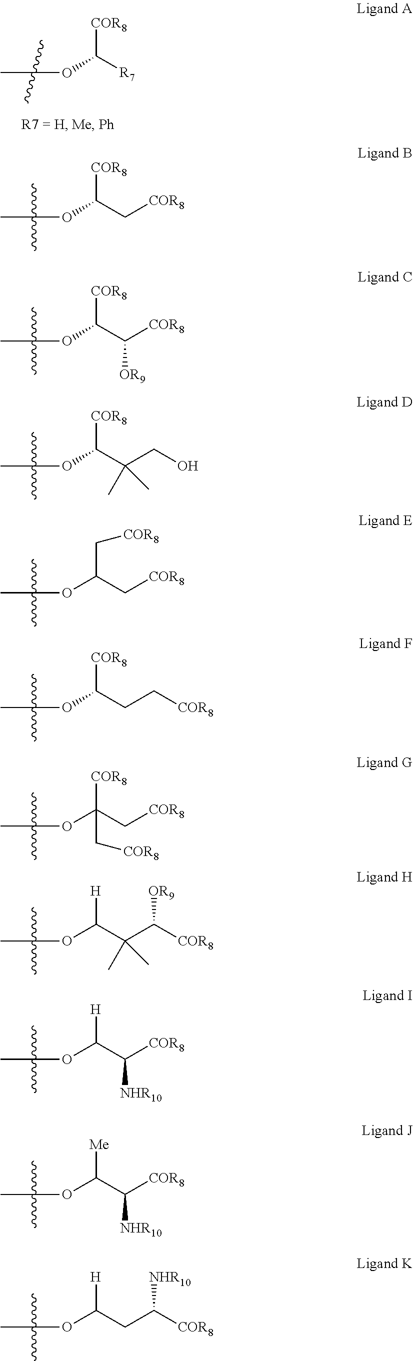

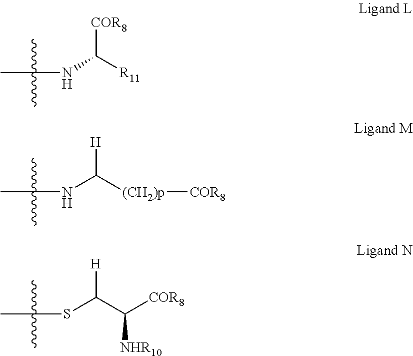

### Wherein,

for ligand A, the lead ligand (the moiety that is attached to the carboxyl group of GHB) is glycolic acid (R7=H), lactic acid (R7=Me, including both enantiomers), mandelic acid (R7=Ph, including both enantiomers),
for ligand B, the lead ligand (the moiety that is attached to the carboxyl group of GHB) is malic acid (including both enantiomers),
for ligand C, the lead ligand (the moiety that is attached to the carboxyl group of GHB) is tartaric acid (including both enantiomers, and the meso-isomer),
for ligand D, the lead ligand (the moiety that is attached to the carboxyl group of GHB) is pantoic acid (attached to the alpha hydroxyl group, including both enantiomers),
for ligand E, the lead ligand (the moiety that is attached to the carboxyl group of GHB) is 3-hydroxy glutaric acid,
for ligand F, the lead ligand (the moiety that is attached to the carboxyl group of GHB) is 2-hydroxy glutaric acid (including both enantiomers),
for ligand G, the lead ligand (the moiety that is attached to the carboxyl group of GHB) is citric acid,
for ligand H, the lead ligand (the moiety that is attached to the carboxyl group of GHB) is pantoic acid (attached to the terminal hydroxyl group, including both enantiomers),
for ligand I, the lead ligand (the moiety that is attached to the carboxyl group of GHB) is the hydroxyl group of amino acid serine,
for ligand J, the lead ligand (the moiety that is attached to the carboxyl group of GHB) is the hydroxyl group of amino acid threonine,
for ligand K, the lead ligand (the moiety that is attached to the carboxyl group of GHB) is the hydroxyl group of amino acid homoserine,
for ligand L, the lead ligand (the moiety that is attached to the carboxyl group of GHB) is the amino group of an amino acid and R11 is the side chain of the lead ligand amino acid,
for ligand M, the lead ligand (the moiety that is attached to the carboxyl group of GHB) is the amino group of a terminal amino acid where p is an integer 0-6, and
for ligand N, the lead ligand (the moiety that is attached to the carboxyl group of GHB) is the sulfhydril group of amino acid cysteine, and,

### R7=H, Me, or Ph

R8=OH, or is an ester formed by the hydroxyl group of another alpha-hydroxy acid or is an amide formed by the amine group of an amino acid, and,
R9=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and
R10=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid.

Upon prodrug cleavage, these GHB prodrugs of Formula A-C will revert back to the original GHB molecule with the hydroxyl group and the carboxyl group present intact.

For the prodrug moiety X, the alpha-hydroxy carboxylic acid and its homo and hetero oligomers (with another alpha-hydroxy carboxylic acid) referred to in this invention should be understood to be covalently bound via a hydroxy group on the alpha-hydroxy carboxylic acid or on the oligomer to another carbonyl (originally part of a carboxyl group of another alpha-hydroxy carboxylic acid, or to another carbonyl of the carboxyl group of the amino acid, or to one carbonyl of the carboxyl group of a dicarboxylic acid (e.g., succinic acid, maleic acid, fumaric acid), while the carboxyl group from the initial alpha-hydroxy carboxylic acid is attached to the GHB.

If the initial carboxyl group that is attached to GHB hydroxyl group referred to in this invention is from a dicarboxylic acid (e.g. malic acid, tartaric acid, citric acid, hydroxy-glutaric acid, succinic acid), it should be understood that the originally formed ester bond can function as ligand formation for structures of Formula A and Formula C, and the second carboxyl group present in the ligand and the hydroxyl group present in the ligand may be further conjugated.

If the initial carboxyl group that is attached to the GHB referred to in this invention is from an amino acid or an acidic amino acid (e.g. aspartic acid, glutamic acid), it should be understood that the amino group of the said amino acid may be bound via a covalent bond as the amide with the carboxyl group on the alpha-hydroxy carboxylic acid or the oligomer carbonyl (originally part of a carboxyl group of the alpha-hydroxy carboxylic acids) or to one carbonyl of the carboxyl group of a dicarboxylic acid (e.g., succinic acid, maleic acid, fumaric acid), or to one carbonyl of the carboxyl group of the fatty acids.

It should also be understood that if the initial carboxyl group that is attached to the GHB referred to in this invention is from alpha-hydroxy carboxylic acids and its homo and hetero oligomers (with another alpha-hydroxy carboxylic acid) to form structures of Formula A and Formula C, the terminal hydroxyl group may be capped as its ester by fatty acids.

It should also be understood that if the initial carboxyl group that is attached to the GHB referred to in this invention is from alpha-hydroxy carboxylic acids and its homo and hetero oligomers (with another alpha-hydroxy carboxylic acid) to form structures of Formula A and Formula C, the terminal hydroxyl group may be capped as its ester by dicarboxylic acids (e.g., succinic acid, maleic acid, fumaric acid).

It should also be understood that if the initial carboxyl group that is attached to the GHB referred to in this invention is from alpha-hydroxy carboxylic acids and its homo and hetero oligomers (with another alpha-hydroxy carboxylic acid) to form structures of Formula A and Formula C, the terminal hydroxyl group may be capped as its ester by amino acids.

For the prodrug moiety Y, when the lead bond is an ester, the alpha-hydroxy carboxylic acid and its homo and hetero oligomers (with another alpha-hydroxy carboxylic acid) referred to in this invention should be understood to be covalently bound via a carboxyl group on the alpha-hydroxy carboxylic acid or on the oligomer to another hydroxyl group (originally part of a hydroxyl group of another alpha-hydroxy carboxylic acid), or to the amine group of the amino acid, while the hydroxyl group from the initial alpha-hydroxy carboxylic acid is attached to the GHB.

For the prodrug moiety Y, when the lead bond is an amide with the amine group of an amino acid, the amino acid referred to in this invention should be understood to be covalently bound via the carboxyl group on the amino acid to another hydroxyl group (originally part of a hydroxyl group of another alpha-hydroxy carboxylic acid, or to another amine group of an amino acid, while the amine group from the initial amino acid is attached to the GHB.

In another embodiment of the present invention, when the covalently modified GHB prodrug compound is provided in oral dosage form (e.g., a tablet, capsule, caplet, liquid dispersion, etc.) it has increased resistance to manipulation. For instance, crushing of a tablet or disruption of a capsule does not substantially increase the rate and amount of GHB absorbed when compositions of the invention are ingested.

In another embodiment of the present invention, when the GHB covalently bound to the prodrug moiety is provided in oral dosage form: for example a tablet, capsule, caplet or other formulation that is resistant to release of GHB by physical manipulation such as crushing.

Another embodiment of the present invention provides GHB prodrug compounds as a composition or method for treating CNS diseases in patients. It should be noted that different GHB prodrug compounds maybe be utilized to treat acute versus chronic conditions.

Another embodiment of the present invention is a composition or method for a sustained-release GHB comprising a covalently bonded GHB prodrug conjugate, wherein said conjugate provides release of GHB at a rate where the level of GHB is within the therapeutic range, but below toxic levels, over an extended period of time (e.g., 8-24 hours or greater).

Another embodiment of the present invention is a composition or method for reducing variability in bioavailability, or preventing a toxic release of GHB, comprising the GHB covalently bonded to the prodrug moiety, wherein said bound GHB maintains a steady-state plasma release curve, which provides therapeutically effective bioavailability but prevents spikes or sharp increases in blood concentrations compared to unbound GHB when given at doses exceeding those that are within the therapeutic range of the GHB.

Another embodiment of the invention is a composition or method for preventing a Cₘₐₓ spike for GHB while still providing a therapeutically effective bioavailability curve comprising a GHB prodrug compound in which GHB has been covalently bonded to a prodrug moiety.

Another embodiment of the present invention is a method for reducing or preventing abuse related to the euphoric effect of a pharmaceutical GHB composition, comprising consuming said composition, wherein said composition comprises a GHB prodrug compound in which a prodrug moiety is covalently attached to GHB, such that the pharmacological activity of GHB is substantially decreased when the composition is used in a manner inconsistent with approved instructions or in a manner that substantially increases the potential of overdose from GHB.

Other embodiments of the present invention are methods wherein said pharmaceutical composition is adapted solely for oral administration, and wherein said GHB is resistant to release from said prodrug moiety when the composition is administered parenterally (e.g., intranasally. intravenously. etc.). Preferably, said GHB would be preferentially released from said prodrug moiety primarily in the presence of acid and/or enzymes present in the stomach or intestinal tract, respectively.

In another embodiment of the present invention, the covalently bonded GHB prodrug may also be in a pharmaceutically acceptable salt form. Pharmaceutically acceptable inorganic and organic acid addition salts are known in the art. Exemplary salts include, but are not limited to, hydrobromide, hydrochloride, hydroiodide, benzoate, bisulfate, tartrate, bitartrate, edetate, edisylate, estolate, esylate, ethanesulfonate, lactate, malate, maleate, mandelate, methanesulfonate, phosphate, 2-hydroxyethanesulfonate, 2-naphthalenesulfonate, 3-hydroxy-2-naphthoate, 3-phenylpropionate, acetate, adipate, alginate, amsonate, aspartate, benzenesulfonate, borate, butyrate, calcium edetate, camphorate, camphorsulfonate, citrate, clavulariate, cyclopentanepropionate, digluconate, dodecylsulfate, finnarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydroxynaphthoate, isothionate, lactobionate, laurate, laurylsulphonate, mucate, naphthylate, napsylate, nicotinate, N-methylglucamine ammonium salt, oleate, palmitate, pamoate, pantothenate, pectinate, phosphateldiphosphate, pivalate, polygalacturonate, propionate, p-toluenesulfonate, saccharate, salicylate, stearate, subacetate, succinate, sulfate, sulfosaliculate, suramate, tannate, teoclate, tosylate, triethiodide, undecanoate, and valerate salts; and inorganic and organic base addition salts that are known in the art such as sodium, potassium, calcium, magnesium, lysine, t-butylamine, choline and the like.

The term “amino acid” refers to one of twenty-two amino acids used for protein biosynthesis, as well as other amino acids that can be incorporated into proteins during translation. Such amino acids can be a natural amino acid, such as glycine, alanine, valine, leucine, isoleucine, aspartic acid, glutamic acid, serine, threonine, glutamine, asparagine, arginine, lysine, proline, phenylalanine, tyrosine, tryptophan, cysteine, methionine, histidine and beta alanine, or non-natural amino acids and alpha amino acids, beta amino acids, gamma amino acids, and epsilon amino acids (e.g., the amino group is remote relative to the carboxyl group).

The present invention also provides methods for providing, administering, prescribing, or consuming a GHB prodrug compound. The invention also provides pharmaceutical compositions comprising a GHB prodrug compound. The formulation of such a pharmaceutical composition can optionally enhance or achieve the desired release profile.

In a further embodiment of the present invention, non-limiting examples of GHB prodrugs of the present invention are shown in Formulae 1-90 and Formulae D-Q and general Formulae R-S. In these formulae, it should be noted that while no salt forms have been depicted, all the formulae compounds can be prepared as their pharmaceutically acceptable salts, as previously described. It should be noted that formulae 1-90 represent GHB prodrug mono-series Formula A, and “GH” represents gamma-hydroxybutyric acid (GHB) and the prodrug component X is chemically/covalently attached to the GHB hydroxyl group via an ester bond. It should also be noted that formulae D-Q represent GHB prodrug mono-series Formula B and the prodrug component Y is chemically/covalently attached to the GHB carboxyl group. In Formulae D-N, attachment is via an ester bond and in Formulae O-Q, attachment is via an amide bond. It should also be noted that general Formulae R-S represent GHB prodrug bis-series Formula C and the prodrug components X and Y are chemically/covalently attached to the GHB molecule through its hydroxyl and carboxyl groups respectively. These compounds may have ester bonds on either end or an ester bond at the hydroxyl terminal and an amide bond at the carboxyl terminal.

Non-limiting examples of GHB prodrugs Formulae 1-90 (representative of Formula A), Formulae D-Q (representative of Formula B) and general Formulae R-S (representative of Formula C) are:

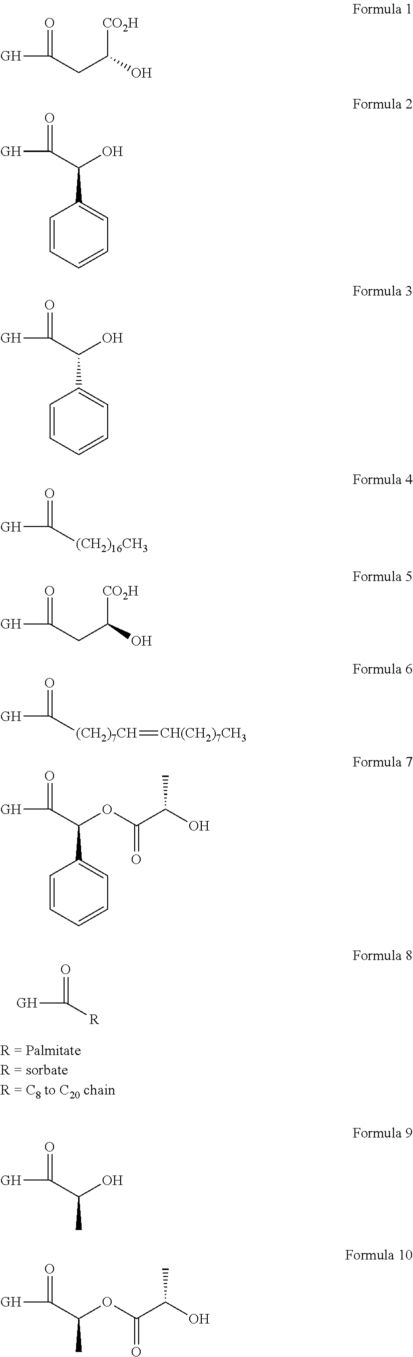

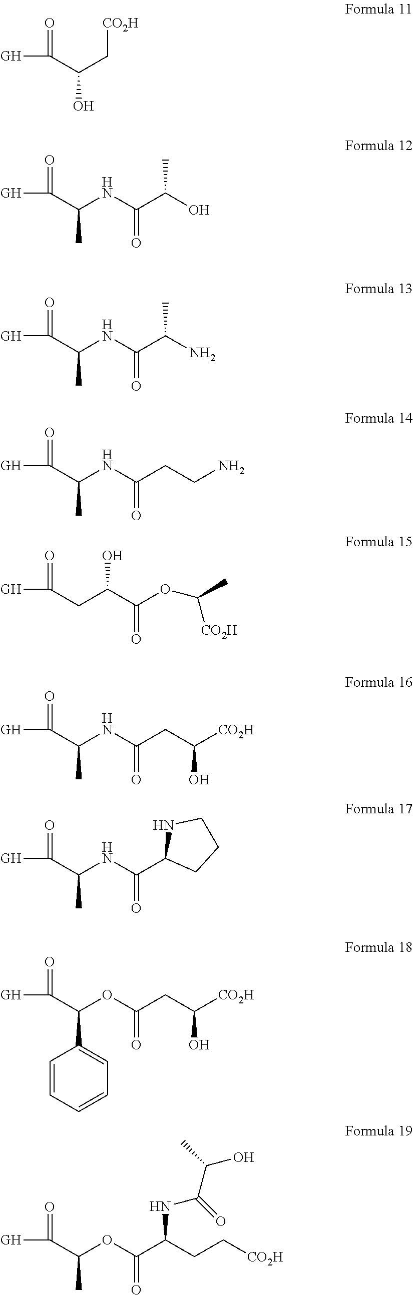

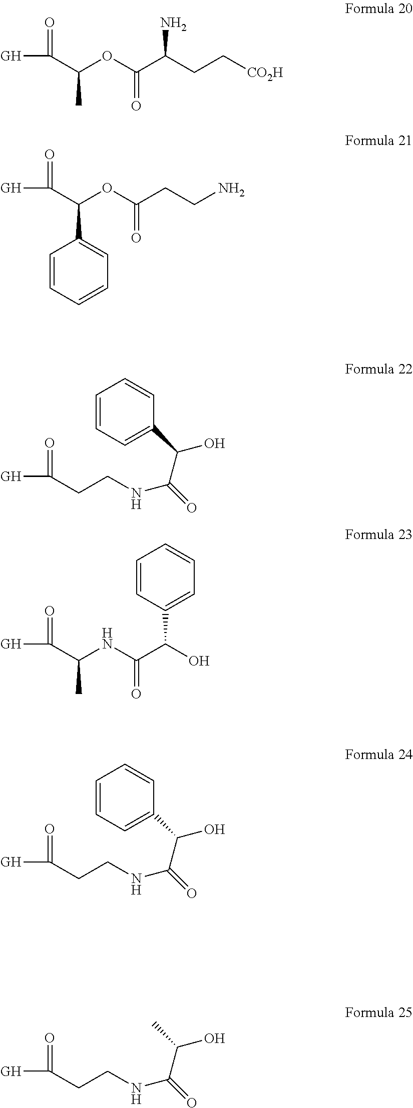

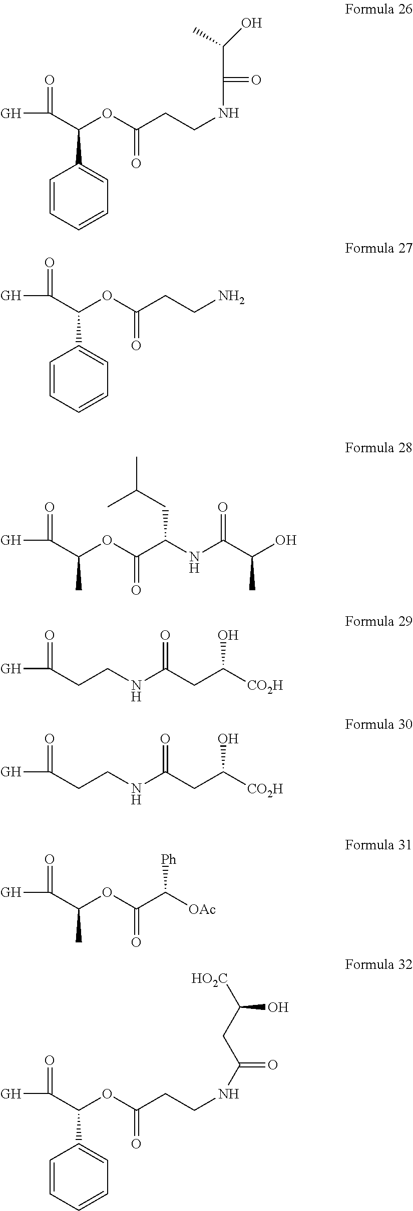

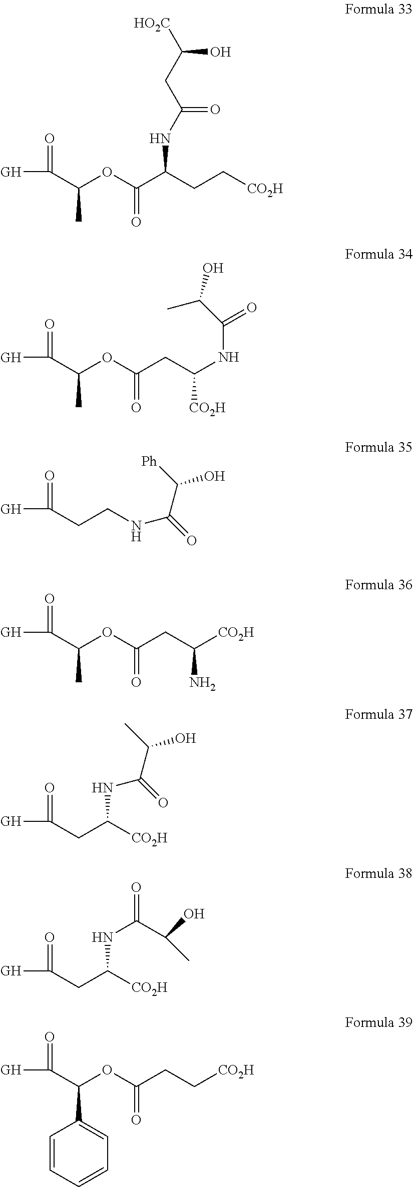

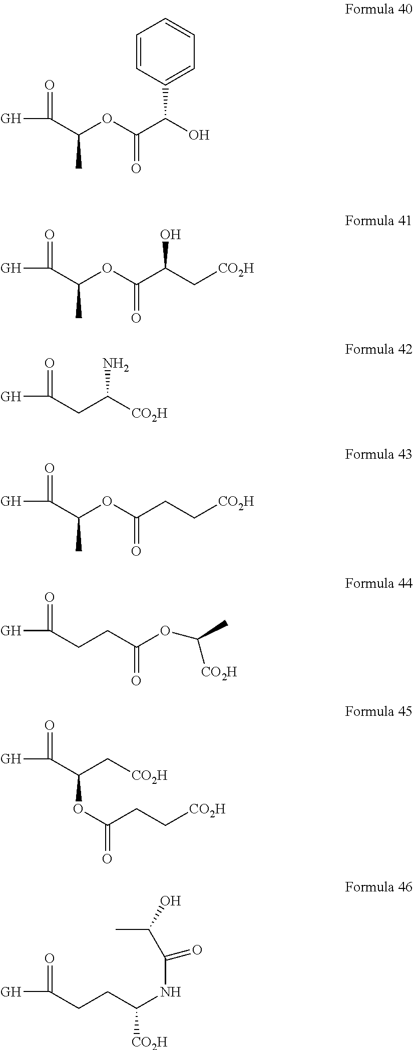

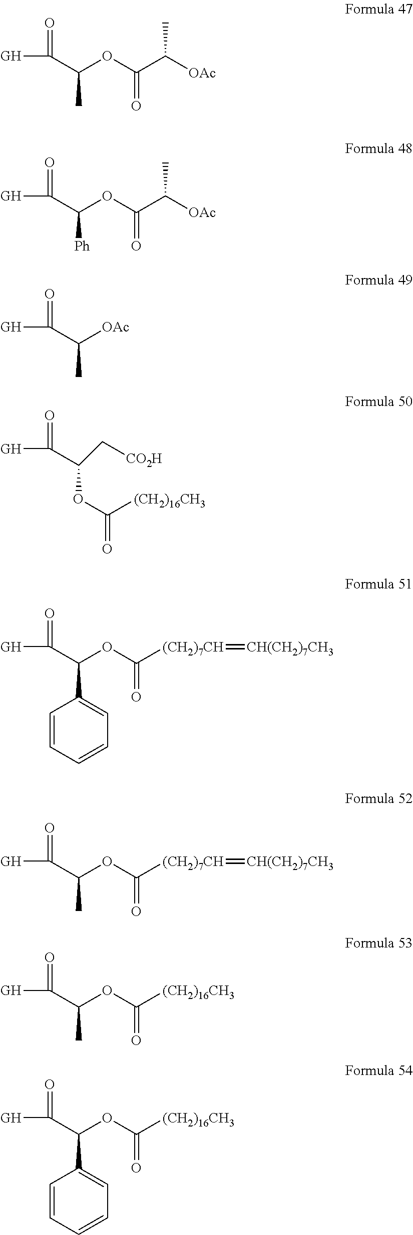

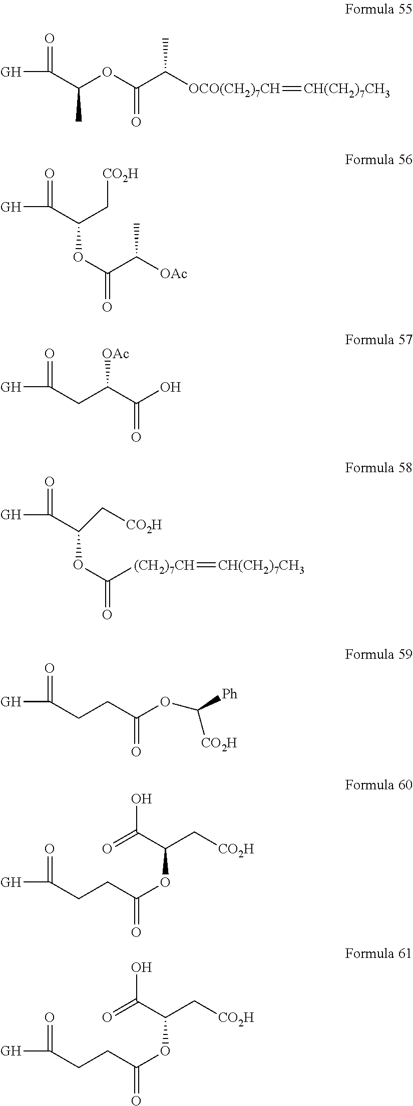

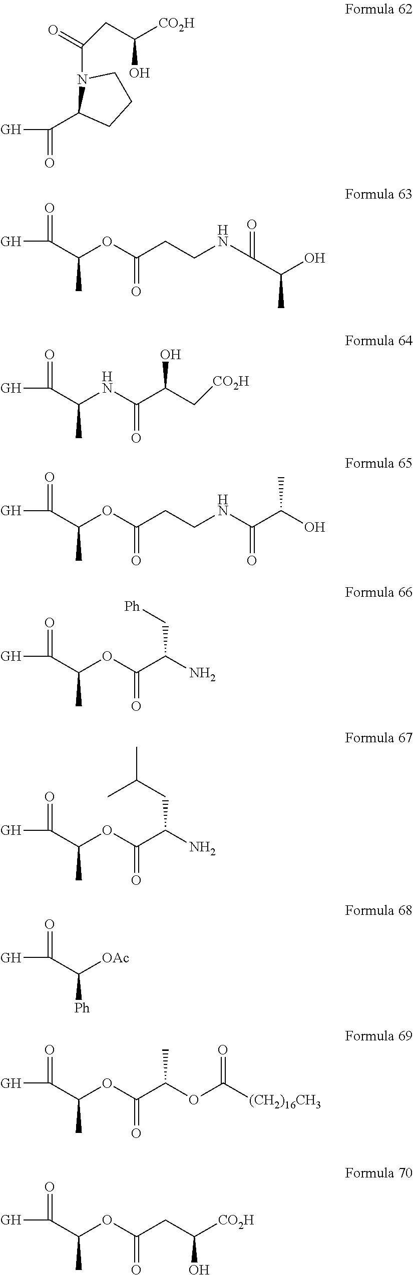

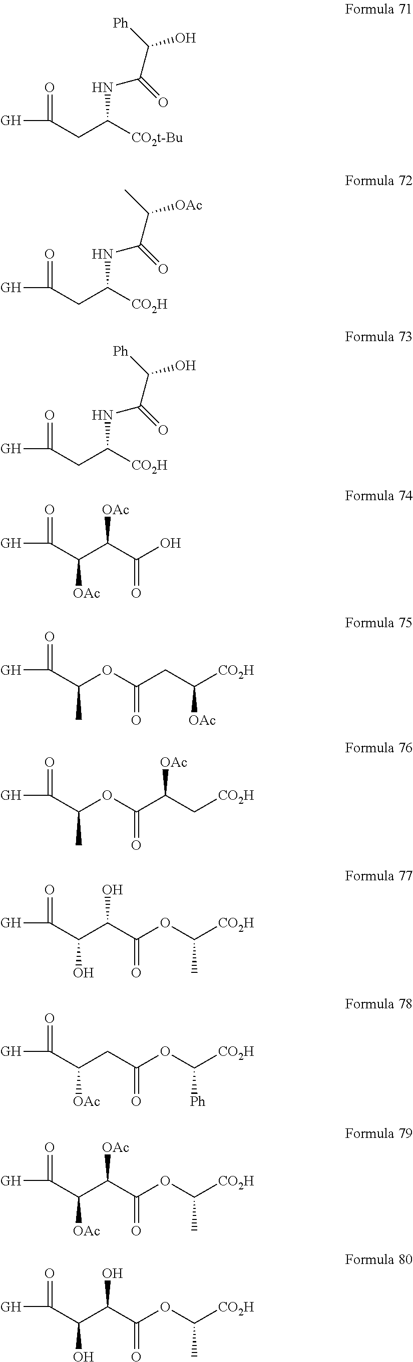

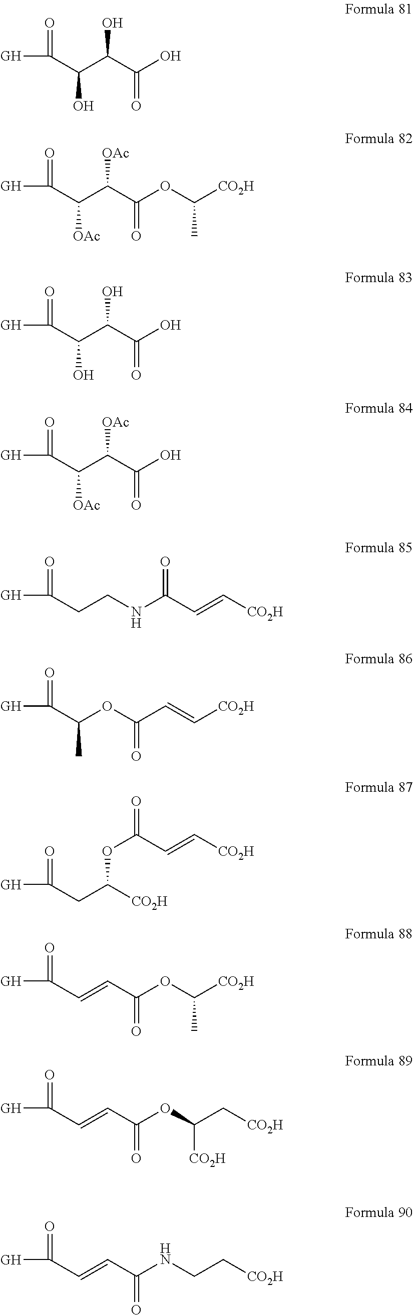

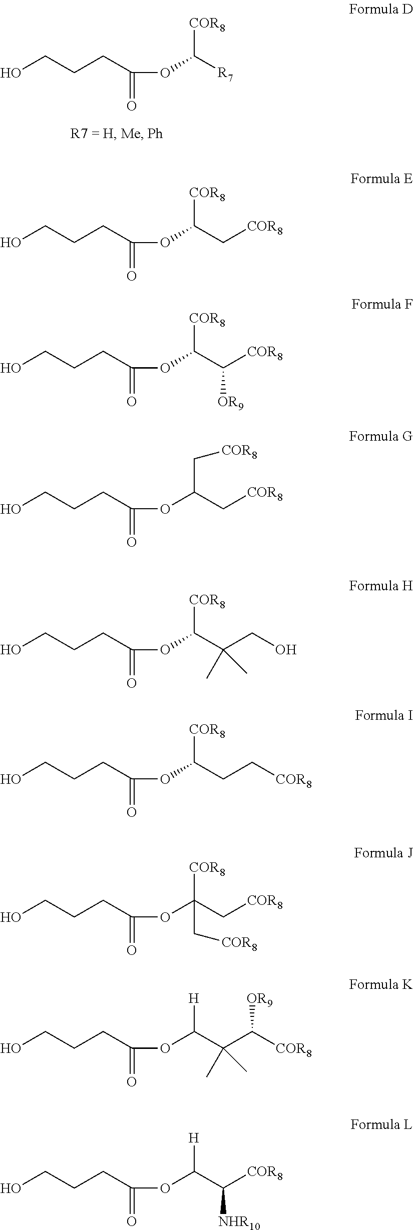

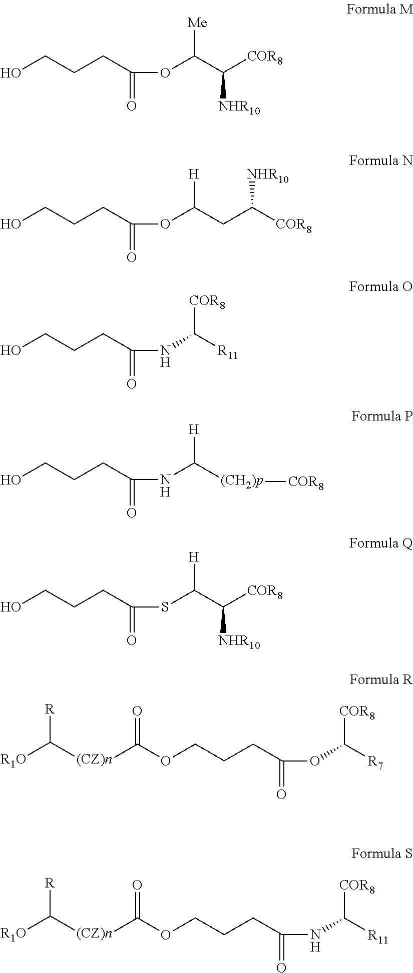

### EXAMPLES

Processes for Preparing GHB Conjugates

General procedures may be used for the preparation of various GHB prodrug conjugates.

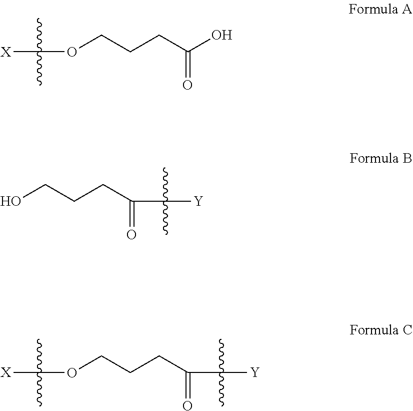

### General Procedure for the Preparation of Conjugates of Formula A:

The procedure involves reacting GHB-t-butyl ester with the carboxyl-activated prodrug moieties.

To a solution of Boc-hydroxyl protected Osu-active ester of alpha-hydroxy acid (1.05 g, 3.4 mmol) in THF (30 mL) is added GHB-t-butyl ester (1.0 eq) and TEA (1.0 eq). The resulting mixture is allowed to stir for 20 h at 20° C. Water (30 mL) is added, and the solution is stirred for 10 minutes prior to removing solvents under reduced pressure. The crude product is dissolved in EtOAc (100 mL) and washed with 2% (aq) AcOH (2×100 mL), saturated NaHCO₃ solution (2×50 mL), and brine (1×100 mL). The organic extract is dried over MgSO₄, filtered, and evaporated to dryness to afford the protected GHB conjugate. This intermediate can be used as is for the next deprotection step or it can be purified by either chromatography or crystallization.

The product is further characterized by nuclear magnetic resonance (NMR) spectroscopy, mass spectroscopy (MS), and elemental analysis.

This procedure will work well with any of the amine bases such as triethylamine (TEA), diisopropyl ethylamine (DIPEA), n-methyl morpholine (NMM).

Boc Group and t-Butyl Group Deprotection from the Coupled GHB Prodrug Product:

Boc group protection is used to protect the hydroxyl group(s) of the alpha-hydroxy carboxylic acids. After the GHB t-butyl ester coupling with the Boc protected (hydroxyl group(s)) of the alpha-hydroxy carboxylic acid, both the t-butyl group and the Boc group are removed by the following general procedure.

To a solution of the hydroxyl Boc-protected and t-butyl ester coupled product (1.5 g) in IPAc (15 mL) is added 4N HClI/dioxane (15 mL) and the reaction mixture is stirred at RT for 2 hours. Water (30 mL) is added, and the solution is stirred for 10 minutes prior to removing solvents under reduced pressure. The crude product is dissolved in EtOAc (100 mL) and washed with 2% (aq) AcOH (2×100 mL) and brine (2×100 mL). The organic extract is dried over MgSO₄, filtered, and evaporated to dryness to afford the protected GHB conjugate. The product is purified by either chromatography or crystallization.

The product is further characterized by nuclear magnetic resonance (NMR) spectroscopy, mass spectroscopy (MS), and elemental analysis.

Deprotection from the Coupled GHB Prodrug Product (Another General Procedure):

Another general procedure may also be used to remove the Boc group and t-butyl ester deprotection from the coupled GHB prodrug product.

To a solution of the above, hydroxyl Boc-protected coupled product (1.5 g) in dichloromethane (15 mL) is added trifluoro acetic acid (15 mL) and the reaction mixture is stirred at RT for 3 hrs. The reaction mixture is concentrated to an oily residue on a rotavap and the residue is further purified by either trituration or chromatography. The product is further characterized by NMR, MS and elemental analysis.

### Synthesis of the Activated Side Chain—OSu Ester for GHB Coupling:

Generally, N-hydroxy succinimide ester activated carboxylic acid of the alpha-hydroxy carboxylic acid is used for stimulant coupling. To a solution of the hydroxyl Boc-protected alpha-hydroxy carboxylic acid (1 g, 1.1 mmol) and NHS (N-hydroxy succinimide) (1.05 eq) in THF (10 mL) is added a solution of DCC (1.05 eq) in THF (5 mL) at 00° C. The reaction mixture is slowly brought to RT and left overnight at RT. The turbid solution is filtered and the filtrate is used as such for the next step coupling process.

Depending on the stability of the specific compound, the —OSu ester can also be precipitated and crystallized.

### General Procedure for the Preparation of Conjugates of Formula B (Ester Series):

Boc-hydroxyl protected GHB acid is converted to its Boc-hydroxyl protected GHB acid —Osu active ester and it is then coupled with alpha-hydroxy acid t-butyl ester.

To a solution of Boc-hydroxyl protected GHB Osu-active ester (2.05 g, 3.4 mmol) in THF (30 mL) is added alpha-hydroxy carboxylic acid t-butyl ester (1.0 eq) and DIPEA (1.0 eq). The resulting mixture is allowed to stir for 20 h at 20° C. Water (30 mL) is added, and the solution is stirred for 10 minutes prior to removing solvents under reduced pressure. The crude product is dissolved in EtOAc (100 mL) and washed with 2% (aq) AcOH (2×100 mL), saturated NaHCO₃ solution (2×50 mL), and brine (1×100 mL). The organic extract is dried over MgSO₄, filtered, and evaporated to dryness to afford the protected GHB conjugate. This intermediate can be used as is for the next deprotection step or it can be purified by either chromatography or crystallization before deprotection.

The product is further characterized by nuclear magnetic resonance (NMR) spectroscopy, mass spectroscopy (MS), and elemental analysis.

For the deprotection, one of the general procedures mentioned above may be used here.

### General Procedure for the Preparation of Conjugates of Formula B (Amide Series):

Boc-hydroxyl protected GHB acid is converted to its Boc-hydroxyl protected GHB acid —Osu active ester and it is then coupled with an amino acid t-butyl ester.

To a solution of Boc-hydroxyl protected GHB Osu-active ester (2.05 g, 3.4 mmol) in THF (30 mL) is added amino acid t-butyl ester (1.0 eq) and NMM (1.0 eq). The resulting mixture is allowed to stir for 10 h at 20° C. Water (30 mL) is added, and the solution is stirred for 10 minutes prior to removing solvents under reduced pressure. The crude product is dissolved in EtOAc (100 mL) and washed with 2% (aq) AcOH (2×100 mL), saturated NaHCO₃ solution (2×50 mL), and brine (1×100 mL). The organic extract is dried over MgSO₄, filtered, and evaporated to dryness to afford the protected GHB conjugate. This intermediate can be used as is for the next deprotection step or it can be purified by either chromatography or crystallization before deprotection.

The product is further characterized by nuclear magnetic resonance (NMR) spectroscopy, mass spectroscopy (MS), and elemental analysis.

For the deprotection, one of the general procedures mentioned above may be used here.

### Another General Procedure for the Preparation of Conjugates of Formula B (Amide Series):

To a solution of gamma-butyrolactone (5.7 gms) in methanol (30 ml) is added the amino acid glycine (7.0 gms) in one portion as a solid and the mixture is refluxed for 20 hours. The solution obtained is evaporated to dryness and the residue obtained is purified either by crystallization or chromatography. As a general procedure, some amino acids may require 10% of water along with methanol to get all the reagents in solution in the beginning of the reaction.

### EMBODIMENTS

Various embodiments are listed below. It will be understood that the embodiments listed below may be combined with all aspects and other embodiments in accordance with the scope of the invention.

### Embodiment 1

GHB prodrugs of the following formulae where the prodrug moieties X and Y are attached covalently to the GHB molecule,

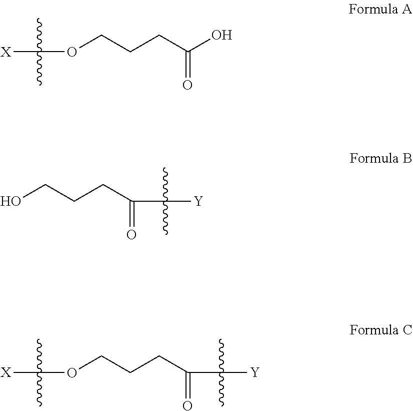

or a pharmaceutically acceptable salt thereof.

### Embodiment 2

GHB prodrugs of embodiments 1 wherein the prodrug moiety X is chemically/covalently attached to the hydroxyl group of GHB as an ester bond and the prodrug moiety Y is chemically/covalently attached to the carboxyl group of GHB as either an ester bond or as an amide bond.

### Embodiment 3

GHB prodrugs of embodiments 1, wherein the prodrug moieties X and Y are same or different and are independent of each other.

### Embodiment 4

GHB prodrugs of embodiments 1, wherein Formula A and Formula B are the mono-derivatives of GHB and Formula C is the bis-derivative of GHB.

### Embodiment 5

The GHB prodrugs of embodiment 1, wherein X is a prodrug moiety (ligand) selected from alpha-hydroxy carboxylic acid and derivatives as monomers, alpha-hydroxy carboxylic acid homo-oligomers, alpha-hydroxy carboxylic acid hetero oligomers with another alpha-hydroxy carboxylic acid, alpha-hydroxy carboxylic acid hetero oligomers with amino acids, alpha-hydroxy carboxylic acid hetero oligomers with dicarboxylic acids, alpha-hydroxy carboxylic acid hetero oligomers with fatty acids, amino acids, fatty acids, and other GRAS-based reagents.

### Embodiment 6

The GHB prodrugs of embodiment 5 wherein homo- and hetero-‘mers’ include linear and branched ‘mers’. The homo- and hetero-‘mers’ may also bevcross linked with other GRAS reagents such as alpha-hydroxy carboxylic acid and amino acids.

### Embodiment 7

The GHB prodrugs of embodiment 5 wherein the alpha-hydroxy carboxylic acid is lactic acid, tartaric acid, malic acid, citric acid, mandelic acid, pantoic acid, pantothenic acid, 2-hydroxy glutaric acid, 3-hydroxy glutaric acid, or other poly-hydroxy carboxylic acids derived from sugars and carbohydrates. The naturally occurring (L)-isomers, the non-natural (D)-isomers, varying mixtures of (L) and (D) isomers, racemates and mixtures of diastereomers, and meso-isomers are all included in this invention.

### Embodiment 8

The GHB prodrugs of embodiment 5 wherein the amino acids include natural (all 22 of the proteinogenic amino acids), and non-natural amino acids, (L)-isomers, (D)-isomers, varying mixtures of (L) and (D) isomers, racemates and mixtures of diastereomers. The amino acids represented here also include alpha amino acids, beta amino acids, gamma amino acids, and epsilon amino acids (amino group remote relative to the carboxyl group).

### Embodiment 9

The GHB prodrugs of embodiment 5 wherein the fatty acids are long chain carboxylic acids, ranging in lengths from eight carbons (C8) to twenty carbons (C20), and said fatty acids include linear and branched chains, and saturated and non-saturated chains, and in the case of unsaturated fatty acids could be either cis- or trans-isomers (Z and E isomers), wherein examples of such fatty acids include, but are not limited to, sorbic acid, stearic acid, oleic acid, palmitic acid, and linoleic acid.

### Embodiment 10

The GHB prodrugs of embodiment 5 wherein the dicarboxylic acids used to make hetero oligomers with alpha-hydroxy carboxylic acid include, but not limited to, fumaric acid, maleic acid, and succinic acid.

### Embodiment 11

The GHB prodrugs of embodiment 1, wherein ligand X is represented as any of ligands 1-16 (shown below);

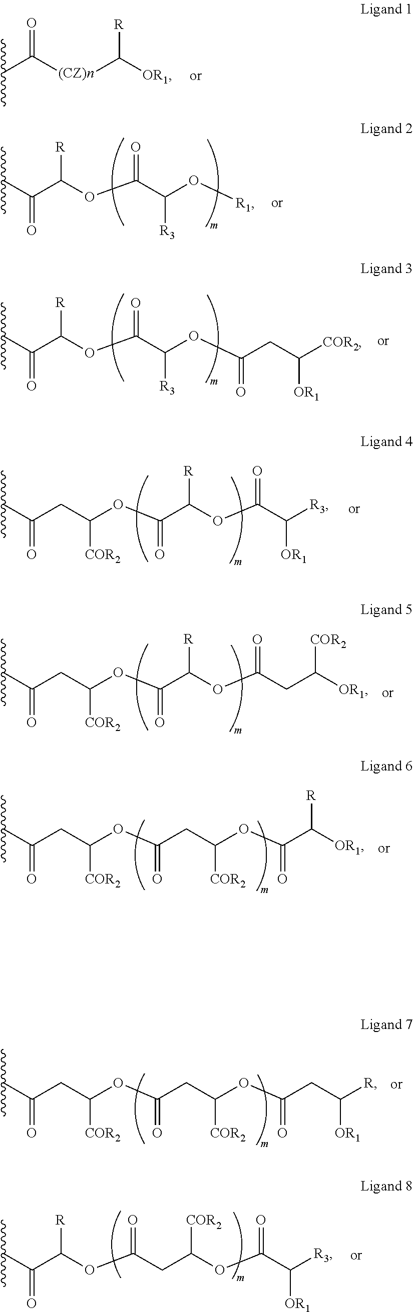

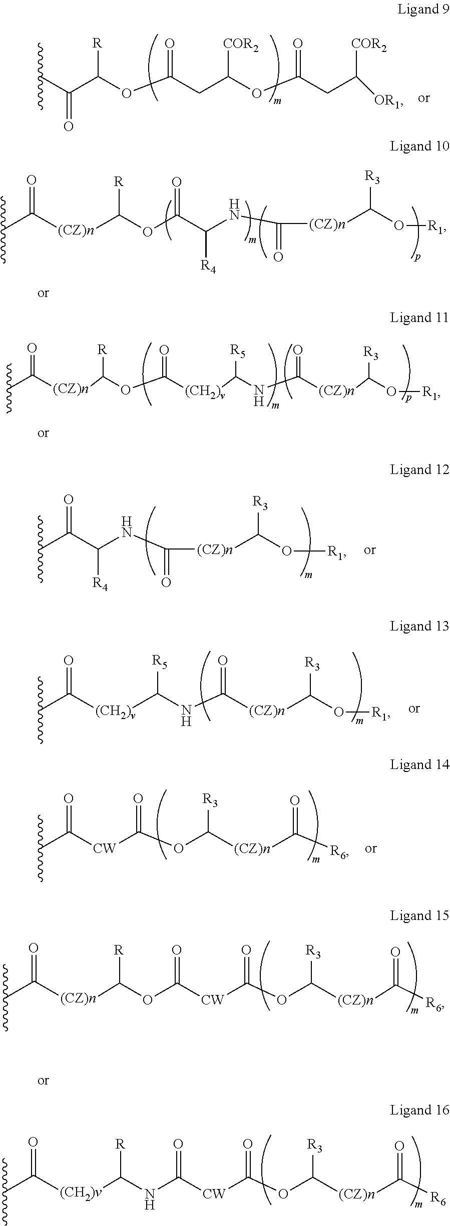

Wherein, in ligands 1-16,

### CZ═CH2, or CHOR1,

R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid,
R=Me, Ph, CH2COR2, CHOR1COR2, or COR2 (when n is not zero),
R2=OH, or is an ester formed by the hydroxyl group of another alpha-hydroxy acid or is an amide formed by the amine group of an amino acid, or is O-alkyl (alkyl esters, where the alkyl group is 1-4 carbon linear or branched, saturated or non-saturated alkyl groups),
R3=Me, Ph, CH2COR2, CHOR1COR2, or COR2 (when n is not zero),
R4 is the side chain of an amino acid, (R4 in ligands 10 and 12),

### R5=H, or COR2,

CW═(CH2)q, or CH═CH (both E and Z isomers),
R6=OH or is an ester formed by the hydroxyl group of another alpha-hydroxy acid or is an amide formed by the amine group of an amino acid, or is alkyl ester, or is an ester with an alkyl group (O-alkyl, alkyl group is 1-4 carbon linear or branched, saturated or non-saturated alkyl groups),
and m is an integer selected from 0 to 4, and n is an integer selected from 0 to 2, and q is an integer selected from 2 to 6, and v is an integer selected from 0 to 6

### Embodiment 12

The GHB prodrugs of embodiment 1, wherein X is a prodrug moiety represented by ligand 17;

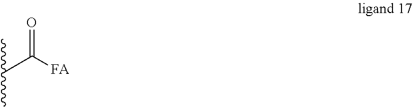

Wherein, FA is C8 to C20 saturated fatty acids, or C8 to C20 unsaturated fatty acids

### Embodiment 13

GHB prodrugs of embodiment 12, wherein FA is sorbic acid, stearic acid, oleic acid, palmitic acid, or linoleic acid.

### Embodiment 14

GHB prodrugs of embodiment 12, wherein FA is linear or branched chain fatty acids, and in the case of unsaturated fatty acids, includes cis- and trans-isomers (Z and E isomers).

### Embodiment 15

GHB prodrugs of embodiment 1, wherein Y is a prodrug moiety ligand, selected from the group consisting of alpha-hydroxy carboxylic acid and derivatives as monomers, alpha-hydroxy carboxylic acid homo-oligomers, alpha-hydroxy carboxylic acid hetero oligomers with another alpha-hydroxy carboxylic acid, alpha-hydroxy carboxylic acid hetero oligomers with amino acid, amino acids, and other GRAS-based reagents.

### Embodiment 16

GHB prodrugs of embodiment 1, wherein Y is a prodrug moiety consisting of any of ligands A-N,

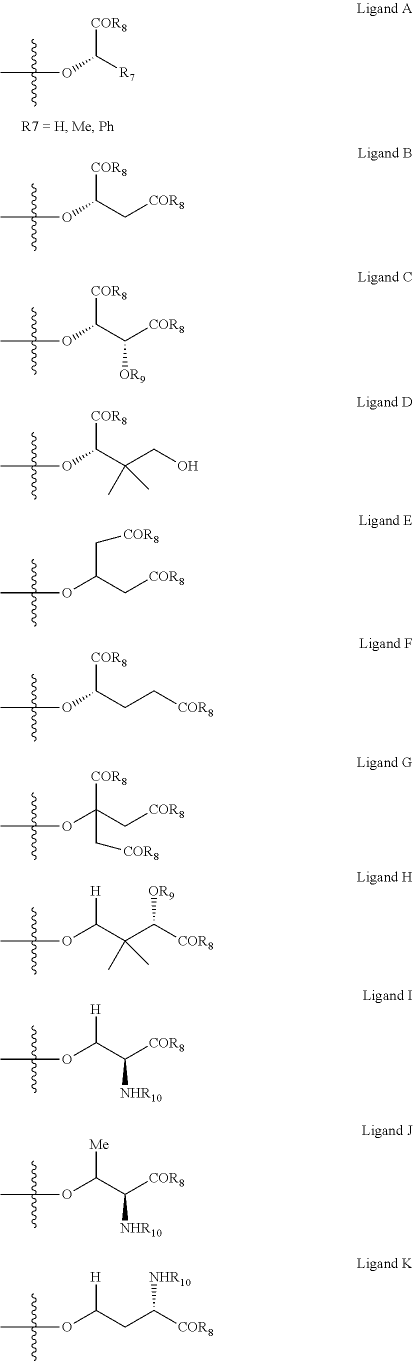

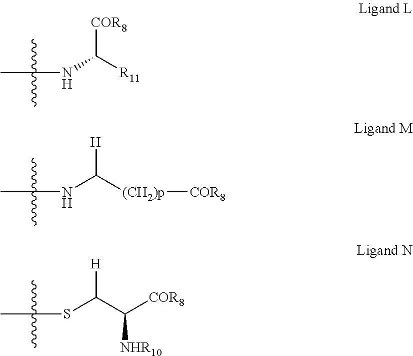

### Wherein,

### R7=H, Me, or Ph

R8=OH, an ester formed by the hydroxyl group of another alpha-hydroxy acid, or is an amide formed by the amine group of an amino acid, and,
R9=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and
R10=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid.

### Embodiment 17

GHB prodrug compounds represented by any one of Formulae 1-90, any one of Formulae D-Q, or any one of general Formulae R-S.

### Embodiment 18

A composition comprising the compound of any of embodiments 1-17

### Embodiment 19

The composition of embodiment 18 wherein the compound or a pharmaceutically acceptable salts thereof maintains a steady-state release curve of GHB in blood that provides therapeutically effective GHB bioavailability.

### Embodiment 20

The composition of embodiment 18, wherein when said composition is administered orally and the bioavailability of GHB is maintained.

### Embodiment 21

A method of treating CNS diseases comprising orally administering the composition of embodiment 18 to a patient.

### Embodiment 22

The pharmaceutical composition of embodiment 18, wherein the compound is a pharmaceutically acceptable salt form.

### Embodiment 23

A pharmaceutical composition comprising one or more of the GHB prodrugs of embodiment 17 and one or more pharmaceutically acceptable excipients.

1. GHB prodrugs of any of the following formulae wherein prodrug moieties X and Y are attached covalently to the GHB molecule, 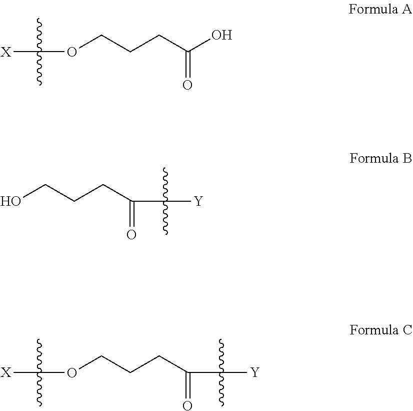
or a pharmaceutically acceptable salt thereof.

2. The GHB prodrugs of claim 1 wherein the prodrug moiety X is chemically/covalently attached to the hydroxyl group of GHB as an ester bond and the prodrug moiety Y is chemically/covalently attached to the carboxyl group of GHB as either an ester bond or as an amide bond.

3. The GHB prodrugs of claim 1 wherein the prodrug moieties X and Y are the same or different.

4. The GHB prodrugs according to claim 1 wherein the prodrug moiety X is a ligand selected from the group consisting of alpha-hydroxy carboxylic acid and derivatives as monomers, alpha-hydroxy carboxylic acid homo-oligomers, alpha-hydroxy carboxylic acid hetero oligomers with another alpha-hydroxy carboxylic acid, alpha-hydroxy carboxylic acid hetero oligomers with amino acid, alpha-hydroxy carboxylic acid hetero oligomers with dicarboxylic acids, alpha-hydroxy carboxylic acid hetero oligomers with fatty acids, amino acids, fatty acids, and other GRAS-based reagents.

5. The GHB prodrugs according to claim 4 wherein homo- and hetero-‘mers’ are linear or branched ‘mers’ or wherein the hetero-‘mers’ are cross linked with other GRAS reagents.

6. The GHB prodrugs according to claim 4 wherein the alpha-hydroxy carboxylic acid is selected from the group consisting of lactic acid, tartaric acid, malic acid, citric acid, mandelic acid, pantoic acid, pantothenic acid, 2-hydroxy glutaric acid, 3-hydroxy glutaric acid, and other poly-hydroxy carboxylic acids derived from sugars and carbohydrates.

7. The GHB prodrugs according to claim 4 wherein the amino acids are selected from the group consisting of any of the 22 naturally occurring proteinogenic amino acids, non-natural amino acids, (L)-isomers, (D)-isomers, mixtures of the (L) and (D) isomers, racemates and mixtures of diastereomers.

8. The GHB prodrugs according to claim 4 wherein the fatty acids are long chain carboxylic acids, ranging in lengths from eight carbons (C8) to twenty carbons (C20).

9. The GHB prodrugs according to claim 4 wherein dicarboxylic acids of the hetero oligomers with alpha-hydroxy carboxylic acid are fumaric acid, maleic acid, or succinic acid.

10. The GHB prodrugs according to claim 1 wherein X is a prodrug moiety selected from of any of ligands 1-16: 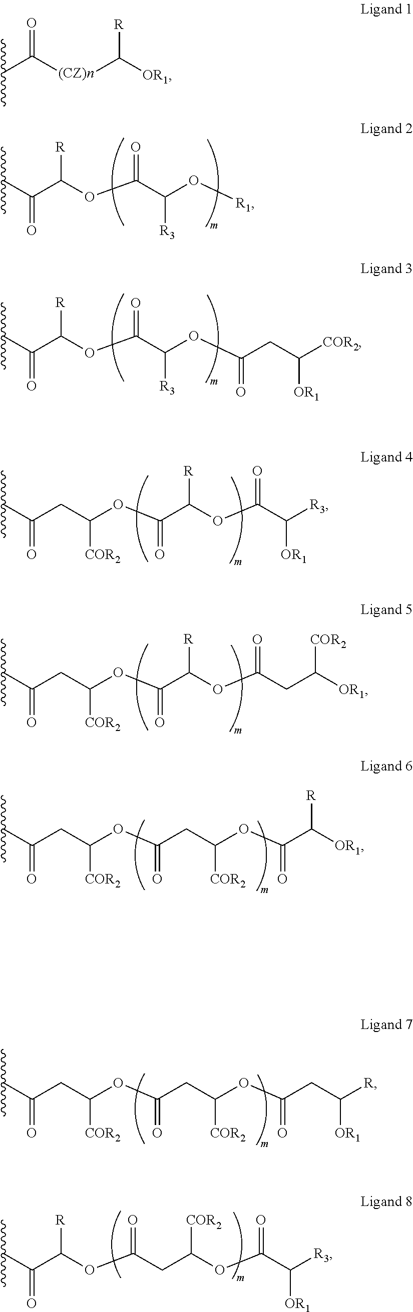 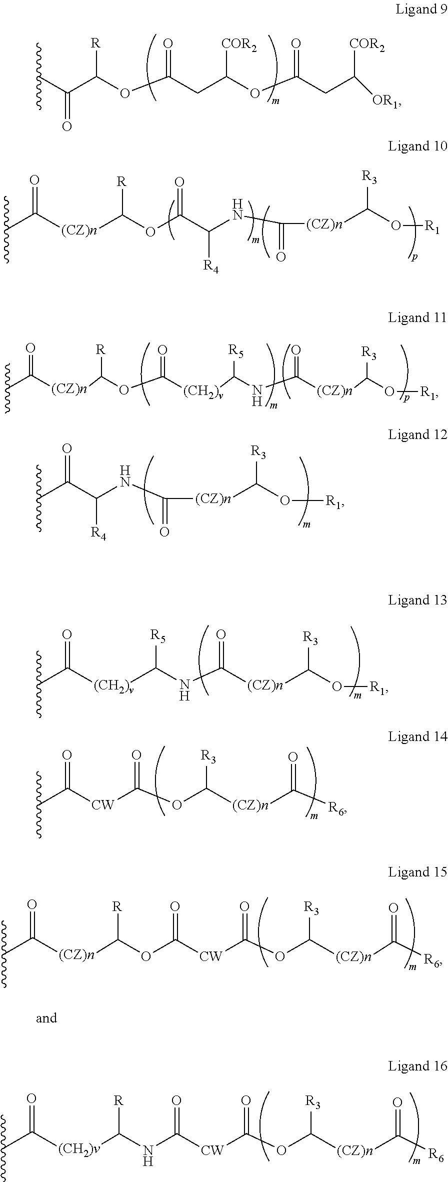
wherein, in ligands 1-16,
CZ═CH2, or CHOR1,
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid,
R=Me, Ph, CH2COR2, CHOR1COR2, or COR2 (when n is not zero),
R2=OH, or is an ester formed by the hydroxyl group of another alpha-hydroxy acid or is an amide formed by the amine group of an amino acid, or is O-alkyl (alkyl ester, where the alkyl group is 1-4 carbon linear or branched, saturated or non-saturated alkyl groups),
R3=Me, Ph, CH2COR2, CHOR1COR2, or COR2 (when n is not zero),
R4 is the side chain of an amino acid,
R5=H, or COR2,
CW═(CH2)q, or CH═CH (both E and Z isomers),
R6=OH or is an ester formed by the hydroxyl group of another alpha-hydroxy acid or is an amide formed by the amine group of an amino acid, or is an ester with an alkyl group (O-alkyl, alkyl group is 1-4 carbon linear or branched, saturated or non-saturated alkyl groups), and m is an integer selected from 0 to 4, and n is an integer selected from 0 to 2, and q is an integer selected from 2 to 6, and v is an integer selected from 0 to 6.

11. The GHB prodrug according to claim 1 wherein the prodrug moiety X is represented by ligand 17: 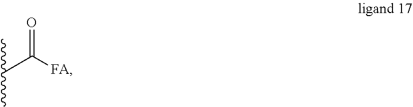
wherein FA is C8 to C20 saturated or unsaturated, linear or branched, fatty acids.

12. The GHB prodrugs according to claim 11, wherein FA is sorbic acid, stearic acid, oleic acid, palmitic acid, or linoleic acid.

13. The GHB prodrugs according to claim 1 wherein the prodrug moiety Y is a ligand selected from the group consisting of alpha-hydroxy carboxylic acid and derivatives as monomers, alpha-hydroxy carboxylic acid homo-oligomers, alpha-hydroxy carboxylic acid hetero oligomers with another alpha-hydroxy carboxylic acid, alpha-hydroxy carboxylic acid hetero oligomers with amino acids, amino acids, and other GRAS-based reagents.

14. The GHB prodrugs according to claim 1 wherein the prodrug moiety Y is selected from any of ligands A-N, 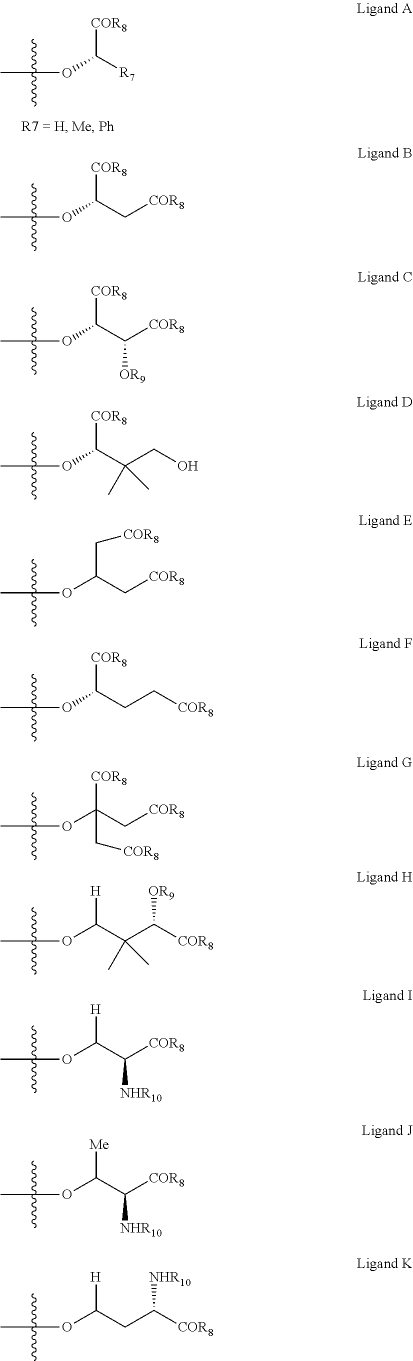 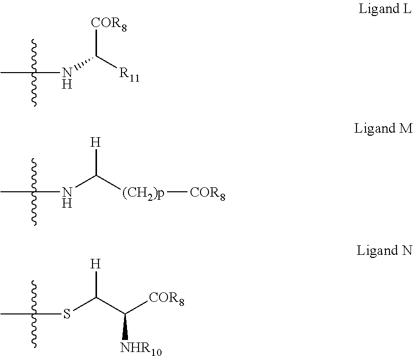
wherein,
R7=H, Me, or Ph
R8=OH, or is part of an ester formed by the hydroxyl group of another alpha-hydroxy acid or is an amide formed by the amine group of an amino acid, and,
R9=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and
R10=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and
R11=a side chain of an amino acid.

15. A GHB prodrug compound represented by any one of Formulae 1-90, Formulae D-Q and general Formulae R-S.

16. A pharmaceutical composition comprising the compound of claim 1 or 2 and a pharmaceutically acceptable excipient.

17. (canceled)

18. The pharmaceutical composition of claim 16, wherein the compound is a pharmaceutically acceptable salt form.

19. A method of treating a CNS disease comprising orally administering the composition of claim 16 to a patient.

20. (canceled)

21. (canceled)
